(19) United States
(12) Patent Application Publication
(10) Pub. No.: US 2019/0324018 A1
(43) Pub. Date: Oct. 24, 2019

(54) SALIVA REAGENT STRIP FOR DETECTING CONCENTRATION OF PEPSIN OR PEPSINOGEN

(71) Applicant: Mark D. Noar, Owings Mills, MD (US)

(72) Inventor: Mark D. Noar, Owings Mills, MD (US)

(21) Appl. No.: 16/386,310

(22) Filed: Apr. 17, 2019

Related U.S. Application Data
(60) Provisional application No. 62/659,728, filed on Apr. 19, 2018.

(51) Int. Cl. G01N 33/52; G01N 33/573

(52) U.S. Cl. CPC G01N 33/525; G01N 33/573

(57) ABSTRACT

A reagent strip determines a concentration of an analyte in a saliva sample. The reagent strip includes a sample receiving portion having a detector reagent thereon. The sample receiving portion includes at least one indicator constructed and arranged to indicate a specific concentration of the analyte in a saliva sample. The strip is constructed and arranged such that when the saliva sample is introduced onto the sample receiving portion, a chemical reaction occurs if the analyte is present in the saliva sample and the indicator will activate if a specific concentration of the analyte is present within the saliva sample so as to indicate the specific concentration. A kit of reagent strips is also disclosed.

### FIELD

The embodiment relates to a saliva reagent strip and, more particularly, to saliva reagent strip for visibly indicating the concentration of pepsin and/or pepsinogen in a salvia sample.

### BACKGROUND

Pepsin is the principle digestive enzyme of the stomach. U.S. Pat. No. 5,879,897, the content of which is hereby incorporated by reference into this specification, discloses a diagnostic method for diagnosing esophageal and extraesophageal gastric reflux (EEGR) and gastroesophageal reflux disease (GERD) by detecting the presence of pepsin of subjects suspected of having a reflux disorder or disease.

U.S. Pat. No. 6,372,513, the content of which is hereby incorporated by reference, discloses a lateral flow test strip for diagnostic testing of oral fluids including saliva. However, such a test strip merely permits the saliva to migrate along the strip, moving the antibody-marker agents along the strip. A chemical reaction will occur only if the analyte is present in the test sample. Thus, a concentration of a particular enzyme in the saliva cannot be obtained or shown on the strip.

CN 105092563 discloses a chemiluminescence ELISA kit for detecting pepsase. However, such detection requires the use of additional equipment such as a microplate reader with chemiluminescence capability (luminometer) to measure the light produced.

Thus, there is a need to provide a reagent test strip to determine and visually show a concentration of pepsin and/or pepsinogen in a saliva sample without the need for additional analysis or equipment.

### SUMMARY

An objective of the embodiment is to fulfill the need referred to above. In accordance with the principles of an embodiment, this objective is achieved by a reagent strip for determining a concentration of an analyte in a saliva sample. The reagent strip includes a sample receiving portion having a detector reagent thereon. The sample receiving portion includes at least one indicator constructed and arranged to indicate a specific concentration of the analyte in a saliva sample. The strip is constructed and arranged such that when the saliva sample is introduced onto the sample receiving portion, a chemical reaction occurs if the analyte is present in the saliva sample and the indicator will activate if a specific concentration of the analyte is present within the saliva sample so as to indicate the specific concentration.

In accordance with another aspect of an embodiment, a kit includes a plurality of reagent strips for determining a concentration of an analyte in a saliva sample. Each reagent strip includes a sample receiving portion having a detector reagent thereon. The sample receiving portion includes an indicator constructed and arranged to indicate a specific concentration of the analyte in a saliva sample. The indicator of each strip is different from indicators of other strips of the plurality of strips for detecting different concentrations the analyte. Each strip is constructed and arranged such that when the saliva sample is introduced onto the conjugate pad, a chemical reaction occurs if the analyte is present in the saliva sample and the indicator will activate if a specific concentration of the analyte is present within the saliva sample.

### BRIEF DESCRIPTION OF THE DRAWINGS

FIG. 1 is a view of a reagent strip in accordance with and embodiment, showing indicators that indicate various concentrations of pepsin from saliva samples.

FIG. 2 shows a kit of reagent strips, each having an indicator for a single, specific concentration of pepsin from a saliva sample.

FIG. 3 shows a kit of reagent strips, each having an indicator for a single, specific concentration of pepsinogen from a saliva sample.

FIG. 4 shows a control kit of reagent strips, with one strip having no indicator active which indicates a negative result and with the other strip showing an indicator active that indicates a positive result.

### DETAILED DESCRIPTION OF THE EXEMPLARY EMBODIMENTS

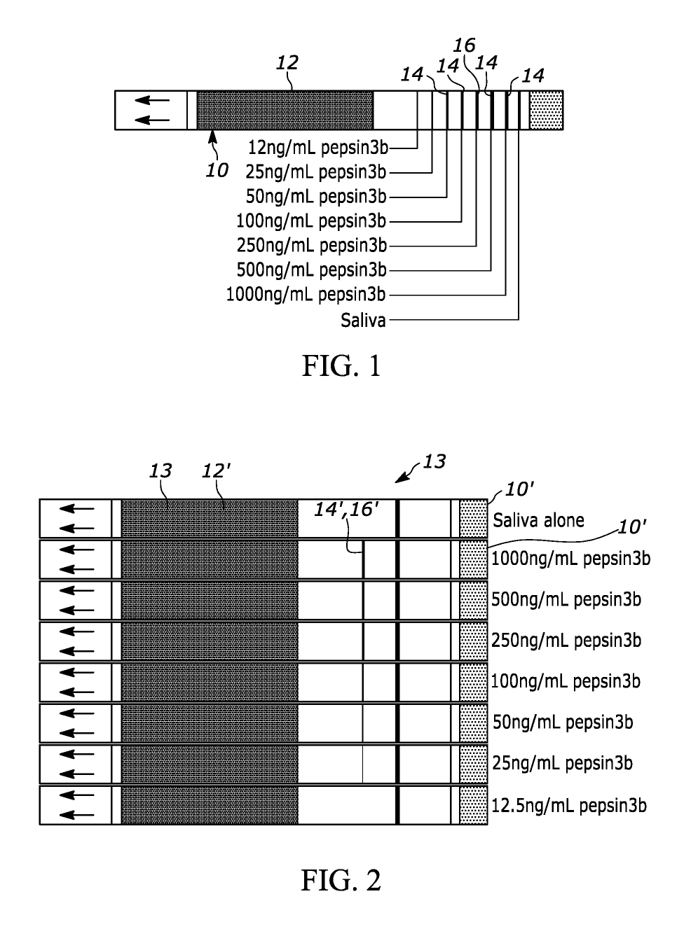

With reference to FIG. 1, a reagent strip, generally indicated at 10, is shown in accordance with an embodiment. The reagent strip 10 may be of the lateral flow test strip type. The reagent strip 10 includes a sample receiving portion 12 having a suitable detector reagent thereon. For example, the detector reagent can be is an antibody agent to detect pepsin or pepsinogen from a saliva sample as known in the art. The sample receiving portion 12 includes a plurality of capture zones 14, each defining an indicator 16 that indicates a different and specific concentration of a target analyte (such as pepsin or pepsinogen) in a saliva sample. In accordance with the embodiment, each capture zone 14 has a specific and different concentration of antibody agent so as to indicate a specific concentration of the target analyte. The process of applying a reagent to a test strip is well-known in the art and may include spraying or jetting the regent(s) onto a nitrocellulose membrane material.

To test a saliva sample with the reagent strip 10, the saliva sample (saliva alone or with a buffer or other amplifier) is introduced onto the sample receiving portion 12 wherein a chemical reaction begins if the analyte is present in the sample. The sample migrates to the capture zones 14 and based on the concentration of the analyte, the corresponding capture zones 14 will activate indicators 16 (vertical lines) that indicate specific concentrations of the analyte within the sample. In the embodiment of FIG. 1, all indicators 16 will be activated, up to the maximum concentration of pepsin within the sample. Thus, as shown in FIG. 1, if the sample has pepsin of 1000 ng/mL pepsin, 1000 ng/mL, 500 ng/mL, 250 ng/mL . . . 12 ng/mL the appropriate pepsin indicators 16 will activate on the strip 10. Alternatively, the strip 10 can be configured with the capture zones 14 so that only a single indicator 16 may be seen on the strip 10 and that indicator 16 will be representative of the weakest level of concentration of the analyte.

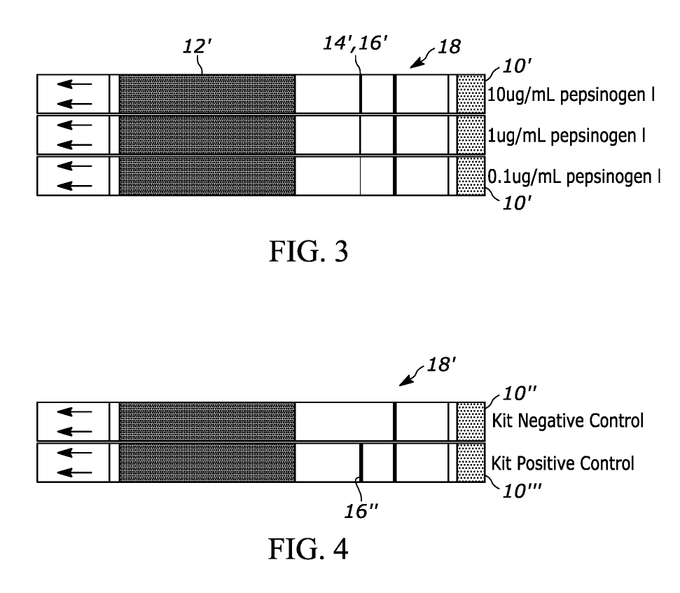

FIGS. 2 and 3 show other embodiments of the invention, each in the form of a kit, generally indicated at 18. Each kit 18 includes a plurality of reagent strips 10′ that are similar to strip 10 having a sample receiving portion 12′ having a suitable detector reagent thereon. However, each strip 10′ of the kit 18 includes only a single indicator 14′ for indicating a specific concentration of a target analyte (such as pepsin in a range between about 12 ng/mL to 100 ng/mL in FIG. 2 or pepsinogen in a range between about 0.1 ug/mL to 10 ug/mL in FIG. 3) in a saliva sample. Thus, when using a kit 18, instead of using a single strip 10 as in FIG. 1, the user would need to use a number of strips 10′ to determine the concentration of the analyte in one saliva sample, since each strip is configure to indicate a single concentration that is different from that of the other strips of the kit.

FIG. 4 shows a control kit 18′ of reagent strips, with one strip 10″ having no indicator active which indicates a negative result and with the other strip 10′″ having an indicator 16″ active, indicating a positive result.

Thus, with the strip 10 or kits 18 of the embodiments, advantageously, concentration of an analyte in a saliva sample is readily visible to a user without the need for additional processing or machines. The ability to obtain a quantitative value of the concentration of pepsin or pepsinogen in a saliva sample with strip 10 or kits 18 enables one to match a disease or symptoms with such concentration.

The foregoing preferred embodiments have been shown and described for the purposes of illustrating the structural and functional principles of the present invention, as well as illustrating the methods of employing the preferred embodiments and are subject to change without departing from such principles. Therefore, this invention includes all modifications encompassed within the spirit of the following claims.

What is claimed is:

1. A reagent strip for determining a concentration of an analyte in a saliva sample, the reagent strip comprising a sample receiving portion having a detector reagent thereon, the sample receiving portion including at least one indicator constructed and arranged to indicate a specific concentration of the analyte in a saliva sample,
wherein the strip is constructed and arranged such that when the saliva sample is introduced onto the sample receiving portion, a chemical reaction occurs if the analyte is present in the saliva sample and the indicator will activate if a specific concentration of the analyte is present within the saliva sample so as to indicate the specific concentration.

2. The reagent strip of claim 1, wherein the sample receiving portion includes a plurality of capture zones, each capture zone corresponding to an indicator, each capture zone having an antibody agent of a concentration that is different from each of the other capture zones, with each indicator being constructed and arranged upon activation, to indicate a different concentration of the analyte when present in the saliva sample.

3. The reagent strip of claim 1, wherein the detector reagent includes an antibody agent to detect pepsin or pepsinogen in the saliva sample.

4. The reagent strip of claim 2, wherein the detector reagent includes an antibody agent to detect pepsin or pepsinogen in the saliva sample.

5. The reagent strip of claim 4, wherein the indicators are constructed and arranged to indicate pepsin in a range of about 12 ng/mL to 1000 ng/mL.

6. A kit comprising:
a plurality of reagent strips for determining a concentration of an analyte in a saliva sample, each reagent strip comprising a sample receiving portion having a detector reagent thereon, the sample receiving portion including an indicator constructed and arranged to indicate a specific concentration of the analyte in a saliva sample, the indicator of each strip being different from indicators of other strips of the plurality of strips for detecting different concentrations the analyte,
wherein each strip is constructed and arranged such that when the saliva sample is introduced onto the conjugate pad, a chemical reaction occurs if the analyte is present in the saliva sample and the indicator will activate if a specific concentration of the analyte is present within the saliva sample.

7. The kit of claim 6, wherein the detector reagent of each reagent strip includes an antibody agent to detect pepsin or pepsinogen in the saliva sample.

8. The kit of claim 6, wherein the indicators are constructed and arranged to indicate pepsin in a range of about 12 ng/mL to 1000 ng/mL.

9. The kit of claim 6, wherein the indicators are constructed and arranged to indicate pepsinogen in a range of about 0.1 ug/mL to 10 ug/mL.
